(19) United States
(12) Patent Application Publication
(10) Pub. No.: US 2020/0245932 A1
(43) Pub. Date: Aug. 6, 2020

(54) SENSORIZED GARMENTS

(71) Applicant: Comftech S.r.l., Monza (MB) (IT)

(72) Inventors: Lara Alessia Laura MOLTANI, Monza (MB) (IT); Luca Domenico ORLANDI, Monza (MB) (IT)

(21) Appl. No.: 16/777,643

(22) Filed: Jan. 30, 2020

(30) Foreign Application Priority Data
Jan. 31, 2019 (IT) 102019000001403

(51) Int. Cl. A61B 5/00; A61B 5/11

(52) U.S. Cl. CPC A61B 5/6803; A61B 5/746; A61B 5/6822; A61B 5/6804; A61B 2562/0261; A61B 5/1112; A61B 5/742; A61B 5/0022; A61B 2562/0247; A61B 5/0053

(57) ABSTRACT

A sensorized garment (1) comprising; an under helmet hood (2) made of a textile material, which can be worn to cover at least the head and neck of an individual; at least one textile pressure sensor (3) and at least one textile deformation sensor (4) incorporated in the under helmet hood (2) and configured to contact the body surface of the individual, each pressure sensor (3) being configured to detect a pressure value to thereby generate a first signal according to the detected pressure value, each deformation sensor (4) being configured to detect a deformation value to thereby generate a second signal according to the detected deformation value; a control unit (5) in signal communication with each pressure sensor (3) and with each deformation sensor (4) for receiving the first signal and the second signal respectively, the control unit (5) being configured to generate an alarm signal when the value of at least one of the first signal and the second signal exceeds a respective preset threshold value.

### FIELD OF THE INVENTION

The present invention relates to a sensorized garment having an under helmet hood for monitoring impacts and deformations occurring by contact with the body surface of a wearer, as defined in the preamble of claim 1.

In particular, the sensorized garment is configured to detect any impact suffered by the wearer, in particular, at the head of the wearer.

### DISCUSSION OF THE RELATED ART

Sensorized garments are known to be provided for detection of the physiological electric signals and vital parameters of an individual.

This sensorized garment comprises suitable textile sensors that can detect the heart rate of the wearer. The heart rate signal associated is stored in a special associated database for future reference.

Prior art sensorized garments are widely used for both medical applications and sports, to monitor the vital parameters of an individual.

A sensor suit is known for sports such as motorcycle racing, which has acceleration sensors and a processing unit that processes the signals generated by the sensors and is thus able to recognize a fall or an imminent impact to immediately deploy the airbags within the suit.

### Problem of the Prior Art

As mentioned above, prior art suits, especially those used in sports applications, afford monitoring of the vital parameters of the individual and actuation of safety systems such as airbags. This is also the case for sports with vehicles such as bicycles, motorcycles, or generally requiring the use of a helmet and protective equipment to limit impacts.

Nevertheless, after an accident, the medical staff that care for the individual have to reconstruct the dynamics of the accident based on the information given by the individual concerned and other witnesses, as well as the overall scene of the accident. Such information may be unreliable as provided by people affected by trauma, even when they simply witness the accident. Furthermore, the reconstruction of the dynamics of the impact only based on the scene of the accident may be inaccurate for its being only theoretical deduced.

### SUMMARY OF INVENTION

The object of the present invention is to provide a sensorized garment that can obviate the drawbacks of the prior art.

A further object of the present invention is to provide a sensorized garment that can provide support to the medical staff after an accident occurred during sports and non-sports activities, and in particular all those activities that require the use of a protective helmet.

The aforementioned technical purpose and objects are substantially fulfilled by a sensorized garment that comprises the technical features as disclosed in one or more of the accompanying claims.

### Advantages of the Invention

One embodiment provides a sensorized garment that can provide the medical staff, especially in first aid situations, with reliable information about the traumas suffered by the person involved in the accident that wears the sensorized garment.

One embodiment provides a sensorized garment that can provide a virtual map for display to the medical staff and represents the amount of traumas that has been suffered by the individual during the accident.

One embodiment provides a sensorized garment that can trigger an alarm signal to request first aid after an accident, providing the medical staff with reliable information about the traumas suffered by the individual involved in the accident.

### BRIEF DESCRIPTION OF THE DRAWINGS

The characteristics and advantages of the present invention will result from the following detailed description of a possible practical embodiment, illustrated as a non-limiting example in the set of drawings, in which:

FIG. 1 shows a sensorized garment of the present invention;

FIG. 2 shows a view of a virtual map generated by means of the sensorized garment of FIG. 1,

FIG. 3 shows one example of application of the sensorized garment of FIG. 1 and of virtual map of FIG. 2 generated thereby,

FIG. 4 shows a side view of a sensorized garment according to a further embodiment of the present invention,

FIG. 5 shows the sensorized garment of FIG. 4 with additional construction details,

FIG. 6 shows a rear view of the of the sensorized garment of FIG. 4,

FIG. 7 shows a front view of a sensorized garment according to a further embodiment of the present invention,

FIG. 8 shows a rear view of the sensorized garment of FIG. 7.

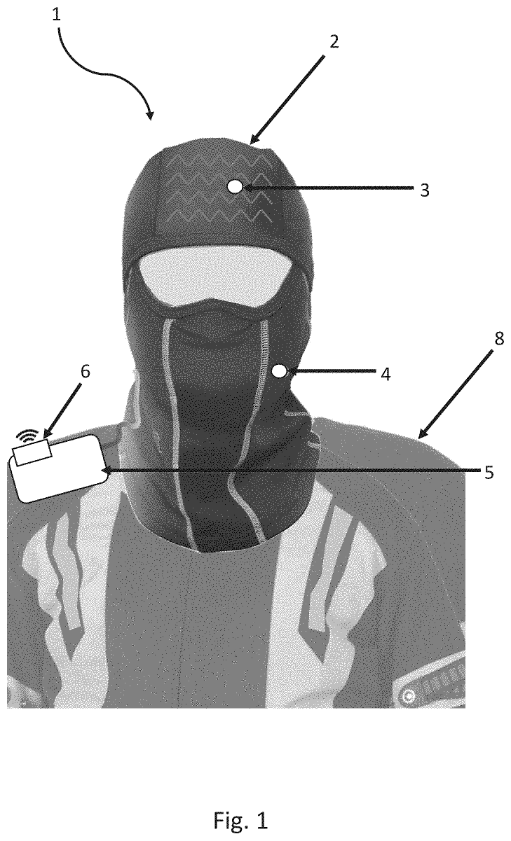

### DETAILED DESCRIPTION

Even when this is not expressly stated, the individual features as described with reference to the particular embodiments shall be intended as auxiliary to and/or interchangeable with other features described with reference to other exemplary embodiments.

The sensorized garment of the annexed figures shall be deemed to be schematically illustrated, not necessarily drawn to scale, and not necessarily representing the actual proportions of its parts.

The present invention relates to a sensorized garment 1 comprising an under helmet hood 2 made of a textile material that can be worn to cover at least the head and neck of an individual, preferably also the shoulders.

As used herein, the term under helmet hood 2 is intended to designate a textile hood having the form of a balaclava, i.e. entirely covering the head and neck, with an opening that leaves the eyes, and optionally also the nose and mouth, exposed, which is used in sports that require the use of a safety helmet. The under helmet hood 2 is adapted to be worn under a protective helmet.

Furthermore, reference is made herein to textile sensorized elements, i.e. having electrical connections incorporated in the textile materials to provide signal communication with the various electronic components, also incorporated in the textile element (FIG. 5). The electronic elements incorporated in the textile element are intended, for example, as the sensors described hereinafter. These sensors and their mutual electrical connections are stably incorporated in the textile element with techniques known in the art such as stitching or molding.

The sensorized garment 1 comprises at least one textile pressure sensor 3 and at least one textile deformation sensor 4 incorporated in the under helmet hood 2. Both the pressure sensor 3 and the deformation sensor 4 are configured to contact the body surface of said individual and are therefore positioned on the under helmet hood 2 to directly interface with the body surface of the individual.

Each pressure sensor 3 is configured to sense a pressure value and generate a first signal in response to the pressure value it has sensed.

Each deformation sensor 4 is configured to sense a deformation value and generate a second signal in response to the deformation value it has sensed.

The sensorized garment 1 comprises a control unit 5 in signal communication with each pressure sensor 3 and each deformation sensor 4 to receive the first signal and the second signal respectively. The control unit 5 is configured to generate an alarm signal when the value of at least one of the first signal and the second signal exceeds a respective preset threshold value. Preferably, the threshold value of the first signal corresponds to the intensity of the compressive force that acts on the body surface beyond which the wearer of the garment 1 would be injured. Likewise, the threshold value of the second signal corresponds to the maximum deformation value for the under helmet hood 2, and hence the maximum elongation of the body surface beyond which the wearer of the sensorized garment 1 would be injured.

Preferably, the control unit 5 is adapted to be removably engaged to the under helmet hood 2. Advantageously, the control unit 5 may be removed and may be used with different under helmet hoods 2.

Advantageously, during operation, the sensorized garment is able to detect compressions and extensions of the wearer's head that might cause traumas and injuries during an impact.

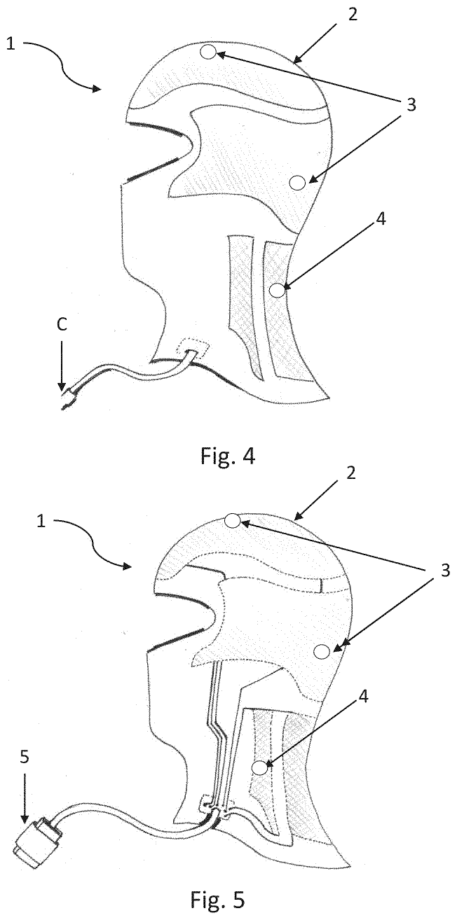

According to the preferred embodiment of the invention, as shown in the FIGS. 4 to 6, each pressure sensor 3 is positioned in a portion of the under helmet hood 2 which is designed to contact one or more regions selected from forehead, scalp and temples of a wearer of the sensorized garment 1.

According to a further preferred embodiment in combination with the previous one, the pressure sensor 3 is positioned in a portion of the under helmet hood 2 that is designed to contact a region selected from nose, jaws, cheeks and cheekbones of a wearer of the sensorized garment 1. By this arrangement the pressure sensor 3 may be advantageously interfaced, during operation, with one of the above listed portions of the body surface of the individual. Thus, when an individual wears the sensorized garment 1, namely the under helmet hood 2 under a helmet, the above listed regions of the wearer's head are most exposed to impact and therefore to compression forces during impacts.

Preferably, the pressure sensor 3 is a piezoelectric sensor.

In the preferred embodiment of the invention, each deformation sensor 4 is positioned in a portion of the under helmet hood 2 that is designed to contact a region of the neck of an individual when he/she wears the sensorized garment 1, and hence the under helmet hood 2. Once again, due to their position, the deformation sensors 4 may sense deformations, namely extensions in the region of the wearer's head that is more exposed to extension during impacts, i.e. the neck.

Preferably, the deformation sensor 4 is a strain gage.

According to a preferred embodiment of the invention, the sensorized garment 1 comprises a plurality of pressure sensors 3 of the aforementioned type, which are configured to generate a plurality of first signals.

Still preferably, the sensorized garment 1 comprises a plurality of deformation sensors 4 of the aforementioned type, which are configured to generate a plurality of second signals.

According to a preferred embodiment of the invention, not shown, the sensorized garment 1 comprises one or more acceleration sensors, preferably an accelerometer. Each acceleration sensor is incorporated into the under helmet hood 2, is configured to detect speed variations to generate a third signal in response to the detected value of speed.

Each acceleration sensor is in signal communication with the control unit 5. Preferably, the control unit 5 is configured to receive the third signal and generate the alarm signal when the value of the third signal exceeds a preset threshold value. Thus, the control unit 5 is able to detect the imminent impact in advance, and determine its severity according to the detected acceleration. Furthermore, the control unit 5 is preferably configured to detect speed variations also after the main impact, to determine whether the individual moved after the accident. This information may be crucial during first aid.

According to a preferred embodiment of the invention, the sensorized garment 1 comprises a transceiver unit 6 associated with the control unit 5 and connected to a mobile network to transmit the alarm signal, the first signal, the second signal and, if any, also the third signal, to an external processing unit 7 connected to the mobile network.

As used herein, the term mobile network is intended to designate a GSM-based telephone network in its various developments such as 3G, 4G, LTE and 5G as well as other data transmission networks such as Wi-Fi, based on protocols known to a skilled person.

Advantageously, the sensorized garment 1 can provide the data collected during an accident to the external processing unit 7, which can process the data to generate a report concerning the dynamics of the impact suffered by the wearer of the sensorized garment 1. It shall be noted that such information reaches the first aid medical staff, who will quickly understand the dynamics of the impact to consider the best strategy for first aid and treatment of the individual involved in the accident.

According to a further preferred embodiment, the transceiver means 6 are configured to connect to a smartphone, a tablet, a PC or a similar external electronic device to transmit the alarm signal, the first signal, the second signal and, if any, also the third signal. These external electronic devices include the external processing unit 7, or transmit the data received from the control unit 5 to a remote external processing unit 7, for example using the aforementioned data transmission networks.

In one aspect, the control unit 5 is configured to generate geolocation data G indicative of the geographic location of the sensorized garment and to send the geolocation data G to the external processing unit 7. The geolocation data G are generated by connection to the mobile network, i.e. the 3G, 4G, LTE and more preferably 5G network.

Alternatively, the geolocation data G may be generated using GPS.

Advantageously, the external processing unit 7 may identify the exact geographic location of the sensorized garment 1, and hence the individual involved in an accident, to thereby be able to provide such geographical coordinates to the first aid mobile units that can quickly identify and reach the wearer of the sensorized garment 1.

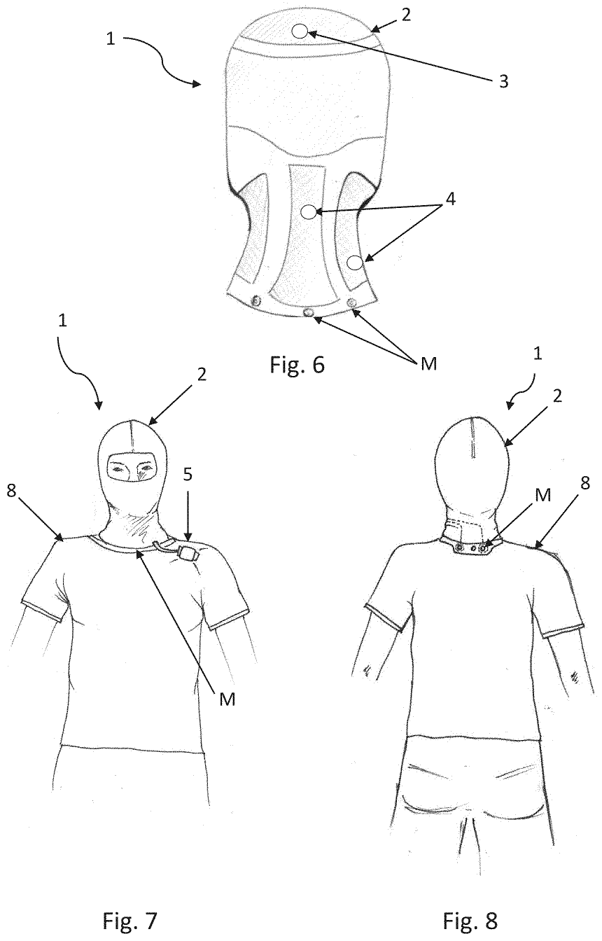

According to a preferred embodiment of the invention, as shown in FIGS. 7 and 8, the sensorized garment comprises a t-shirt 8 made of textile material and adapted to be worn by an individual. The t-shirt 8 and the under helmet hood 2 are connected at least at their respective neck portions by fastening means M, preferably one or more of seams, zippers, snap fasteners, Velcro or press studs. Advantageously, the connection between these two textile elements, i.e. the under helmet hood 2 and the t-shirt 8, will afford improved operation of the deformation sensors 4, especially in elongation detection. This is because the t-shirt 8 can always keep the neck portion of the under helmet hood 2 in position, thereby preventing any slipping that might occur when the hood 2 is stretched and that might alter extension detection or even cause slipping of the under helmet hood 2 off the wearer's head.

Preferably, at least one pressure sensor 3, at least one deformation sensor 4 and optionally at least one acceleration sensor, are also incorporated in the t-shirt 8 to provide the same measurements as described above.

According to a preferred embodiment of the invention, the control unit 5 transmits the first signals, the second signals and optionally the third signals to the external processing unit 7 for a preset duration of time in response to the first event in which the threshold value of at least one of the aforementioned signals is detected to be exceeded, i.e. Upon generation of an alarm signal.

According to a preferred embodiment, the sensorized garment 1 comprises a memory unit (not shown). Preferably, the control unit 5 stores all the first, second and optionally third signals generated during a preset interval of time after the threshold value of at least one of these signals has been exceeded, i.e. after the generation of an alarm signal, in the memory unit.

Advantageously, the sensorized garment 1 tracks all compression, extension and optionally acceleration events occurring during a preset interval of time upon generation of an alarm signal. In other words, the compression, extension and acceleration values are detected and sent to an external unit over a period of time following the accident. This will provide a complete picture of the traumatic events suffered by the wearer of the sensorized garment. In particular, the detection of speed variations after the main impact by means of one or more acceleration sensors, provides information about any movements of the person after the accident. This information is particularly useful for the first aid medical staff to ascertain the actual health conditions of the individual after the accident.

Preferably, the sensorized under helmet hood is equipped with a battery unit for power supply to all of its components.

Advantageously, the sensorized garment 1 is compact, comfortable, hygienic, breathable and easy to wash.

Advantageously, the control unit is actuated as soon as the garment is fastened and remains silent until the time in which an alarm signal is generated and sent to the external processing unit 7. This involves considerable energy savings.

The present invention also relates to a sensorized system and has the purpose to detect and map the impacts that the wearer of the inventive sensorized garment suffers.

For this purpose, the sensorized system comprises the sensorized garment 1 having a plurality of pressure sensors 3 of the aforementioned type, which are configured to generate a plurality of first signals. Also, the sensorized garment 1 comprises a plurality of deformation sensors 4 of the aforementioned type, which are configured to generate a plurality of second signals. Optionally, the sensorized garment 1 comprises a plurality of acceleration sensors of the aforementioned type, which are configured to generate a plurality of third signals.

The sensorized system also comprises an external processing unit 7 in signal communication with the sensorized garment 1.

The control unit 5 is configured to transmit the alarm signal, the plurality of first signals and the plurality of second signals and optionally also the plurality of third signals to the external processing unit 7.

The external processing unit 7 is configured to process the plurality of first signals to generate first map data representative of a first map of the pressure values detected by the plurality of pressure sensors 3 on the surface of said under helmet hood 2.

The external processing unit 7 is configured to process said plurality of second signals to generate second map data representative of a second map of the deformation values detected by the plurality of deformation sensors 4 on the surface of the under helmet hood 2.

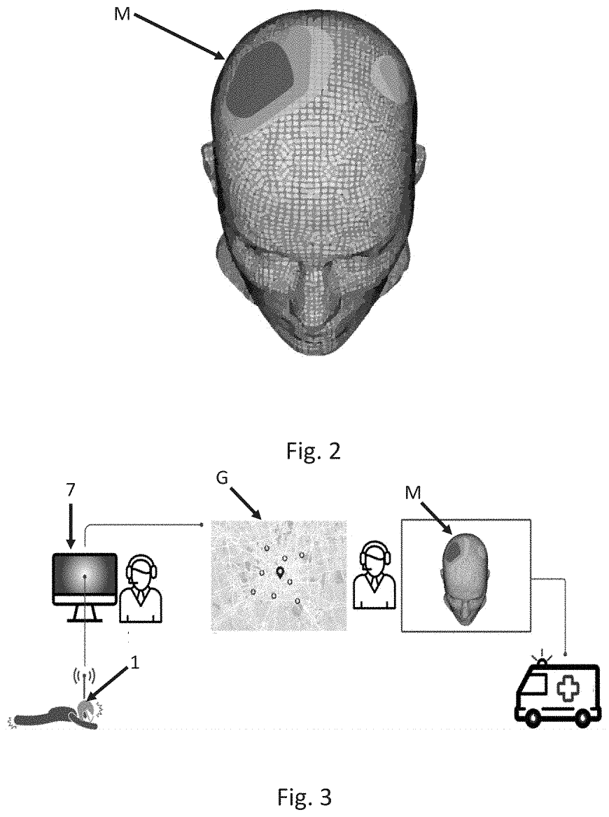

The external processing unit 7 is configured to combine the first map data and the second map data to generate a virtual image map M representative of the detected deformation and compression values and their distribution on the surface of the under First data helmet hood 2. Preferably, the virtual map image M is a three-dimensional map or composed of one or more two-dimensional images, which provide a visual indication of the areas of the sensorized garment 1 that have suffered the impact or impacts, i.e. the compressions and/or deformations. More preferably, the virtual image map M provides a visual indication of the distribution and value of the compressions and deformations on the surface of the sensorized garment 1, and particularly on the surface of the under helmet hood 2 and the t-shirt 8 (if any). Such visual indication is preferably obtained with the definition of colored areas. The extent of each colored area indicates the region of the head affected by compressions and/or deformations, whereas the intensity of the color is related to the intensity of the compression and/or deformation values that have been detected. Advantageously, the virtual image map M as shown in FIGS. 2 and 3 may be provided to the first aid medical staff and is thus useful in diagnosing the individual involved in the accident.

1. A sensorized garment (1) characterized in that it comprises:
an under helmet hood (2) made of a textile material, which can be worn to cover at least the head and neck of an individual;
at least one textile pressure sensor (3) and at least one textile deformation sensor (4) incorporated in said under helmet hood (2) and configured to contact the body surface of said individual, each pressure sensor (3) being configured to detect a pressure value to thereby generate a first signal according to said detected pressure value, each deformation sensor (4) being configured to detect a deformation value to thereby generate a second signal according to said detected deformation value;
a control unit (5) in signal communication with said at least one textile pressure sensor (3) and with said at least one textile deformation sensor (4) for receiving said first signal and said second signal respectively, said control unit (5) being configured to generate an alarm signal when the value of at least one of said first signal and said second signal exceeds a respective preset threshold value;
each textile pressure sensor (3) is placed in a portion of said under helmet hood (2) which is designed to contact one or more areas selected from forehead, scalp, temples, nose, jaws, cheeks and cheekbones of said individual.

2. A sensorized garment (1) as claimed in claim 1, wherein each textile deformation sensor (4) is placed in a portion of said under helmet hood (2) which is designed to contact an area of the neck of said individual.

3. A sensorized garment (1) as claimed in claim 1, comprising a transceiver unit (6) associated with said control unit (5) and connected to a mobile network to transmit said alarm signal, said first signal and said second signal to an external processing unit (7) connected to said mobile network.

4. A sensorized garment (1) as claimed in claim 1, wherein said control unit (5) is configured to generate geolocation data (G) indicative of the geographic location of said sensorized garment (1) and to send said geolocation data (G) to said external processing unit (7).

5. A sensorized garment (1) as claimed in claim 1, wherein said textile pressure sensor (3) is a piezoelectric sensor.

6. A sensorized garment (1) as claimed in claim 1, wherein said textile deformation sensor (4) is a strain gage.

7. A sensorized garment (1) as claimed in claim 1, comprising a t-shirt (8) made of textile material which can be worn by said individual, said t-shirt (8) and said under helmet hood (2) being connected to each other at least at their respective neck portions.

8. A sensorized garment (1) as claimed in claim 7, wherein said at least one textile pressure sensor (3) and at least one textile deformation sensor (4) are also incorporated in said t-shirt (8).

9. A sensorized system for detecting and mapping impacts, comprising:
a sensorized garment (1) comprising an under helmet hood (2) made of a textile material, which can be worn to cover at least the head and neck of an individual;
at least one textile pressure sensor (3) and at least one textile deformation sensor (4) incorporated in said under helmet hood (2) and configured to contact the body surface of said individual, each pressure sensor (3) being configured to detect a pressure value to thereby generate a first signal according to said detected pressure value, each deformation sensor (4) being configured to detect a deformation value to thereby generate a second signal according to said detected deformation value;
a control unit (5) in signal communication with said at least one textile pressure sensor (3) and with said at least one textile deformation sensor (4) for receiving said first signal and said second signal respectively, said control unit (5) being configured to generate an alarm signal when the value of at least one of said first signal and said second signal exceeds a respective preset threshold value;
each textile pressure sensor (3) is placed in a portion of said under helmet hood (2) which is designed to contact one or more areas selected from forehead, scalp, temples, nose, jaws, cheeks and cheekbones of said individual;
an external processing unit (7) in signal communication with said sensorized garment (1);
said system being characterized in that:
said at least one textile pressure sensor (3) comprises a plurality of textile pressure sensors (3) configured to generate a plurality of first signals;
said at least one textile deformation sensor (4) comprises a plurality of deformation sensors (4) configured to generate a plurality of second signals;
said control unit (5) is configured to transmit said alarm signal, said plurality of first signals and said plurality of second signals to said external processing unit (7);
said external processing unit (7) is configured to process said plurality of first signals to generate first map data representative of a first map of the pressure values detected by said plurality of pressure sensors (3) on the surface of said under helmet hood (2);
said external processing unit (7) is configured to process said plurality of second signals to generate second map data representative of a second map of the deformation values detected by said plurality of deformation sensors (4) on the surface of said under helmet hood (2);
said external processing unit (7) is configured to combine said first map data and said second map data to generate the image of a virtual map (M) representative of the detected deformation and compression values and their distribution on the surface of said under helmet hood (2).
